(19) United States
(12) Patent Application Publication
(10) Pub. No.: US 2007/0185373 A1
(43) Pub. Date: Aug. 9, 2007

(54) Gastric band introduction device

(75) Inventor: Mark Tsonton, Loveland, OH (US)

Correspondence Address: WELSH & FLAXMAN LLC, 2000 DUKE STREET, SUITE 100, ALEXANDRIA, VA, 22314, US

(73) Assignee: Ethicon Endo-Surgery, Inc., Cincinnati, OH (US)

(21) Appl. No.: 11/346,592

(22) Filed: Feb. 3, 2006

(51) Int. Cl. A61F 2/00

(52) U.S. Cl. 600/37

(57) ABSTRACT

A gastric band introduction device includes an elongated support tube having a handle at a proximal end thereof and an opening for delivery of a gastric band at a distal end thereof. The introduction device also includes a transparent section formed at the distal end of the support tube. A method for the application of a gastric band through the utilization of an gastric band introduction device is achieved by removing the gastric band introduction device from its packaging, initiating a check of the gastric band prior to introduction within the patient, the check including viewing of the gastric band through a transparent section formed in the introduction device, and introducing the gastric band.

### BACKGROUND OF THE INVENTION

1. Field of the Invention

The present invention relates to an introduction device for gastric bands. In particular, the invention relates to an introduction device including a transparent distal end permitting viewing of the gastric band housed therein.

2. Description of the Prior Art

Morbid obesity is a serious medical condition. In fact, morbid obesity has become highly pervasive in the United States, as well as other countries, and the trend appears to be heading in a negative direction. Complications associated with morbid obesity include hypertension, diabetes, coronary artery disease, stroke, congestive heart failure, multiple orthopedic problems and pulmonary insufficiency with markedly decreased life expectancy. With this in mind, and as those skilled in the art will certainly appreciate, the monetary and physical costs associated with morbid obesity are substantial. In fact, it is estimated the costs relating to obesity are in excess of 100 billion dollars in the United States alone.

A variety of surgical procedures have been developed to treat obesity. The most common currently performed procedure is Roux-en-Y gastric bypass (RYGB). This procedure is highly complex and is commonly utilized to treat people exhibiting morbid obesity. Other forms of bariatric surgery include Fobi pouch, bilio-pancreatic diversion, and gastroplastic or “stomach stapling”. In addition, implantable devices are known which limit the passage of food through the stomach and affect satiety.

In view of the highly invasive nature of many of these procedures, efforts have been made to develop less traumatic and less invasive procedures. Gastric-banding is one such procedure. Gastric-banding is a type of gastric reduction surgery attempting to limit food intake by reducing the size of the stomach. In contrast to RYGB and other stomach reduction procedures, gastric-banding does not require the alteration of the anatomy of the digestive tract in the duodenum or jejunum.

Since the early 1980s, gastric bands have provided an effective alternative to gastric bypass and other irreversible surgical weight loss treatments for the morbidly obese. Several alternative procedures are performed under the heading of gastric-banding. Some banding techniques employ a gastric ring, others use a band, some use stomach staples and still other procedures use a combination of rings, bands and staples. Among the procedures most commonly performed are lap band, vertical banded gastroplasty (VBG), silastic ring gastroplasty (SRG), and adjustable silastic gastric banding (AGB).

In general, the gastric band is wrapped around an upper portion of the patient's stomach, forming a stoma that is less than the normal interior diameter of the stomach. This restricts food passing from an upper portion to a lower digestive portion of the stomach. When the stoma is of an appropriate size, food held in the upper portion of the stomach provides a feeling of fullness that discourages overeating.

Typically, the gastric band is laparoscopically introduced into a patient's abdomen by pushing it through a trocar. Since inserting the gastric band directly through a trocar may also undesirably damage the gastric band, delivery devices have been developed wherein the gastric band is preloaded with the delivery device and is ready for implantation without worrying that the gastric band may contact an exterior skin surface or become damaged as it is moved through the trocar.

Despite the many advantages associated with delivery instruments employing a preloaded gastric band, the recent development of balloon-type gastric bands necessitates various checks prior to insertion of the delivery instrument, for example, it is now often considered desirable to leak test balloon-type gastric bands prior to insertion into the abdominal cavity for implantation. As such, a need currently exists for improved gastric band delivery systems providing for convenient and reliable delivery of gastric bands, including techniques permitting testing of the gastric bands prior to implantation. The present invention provides such a gastric band delivery technique.

### SUMMARY OF THE INVENTION

It is, therefore, an object of the present invention to provide a gastric band introduction device which includes an elongated support tube having a handle at a proximal end thereof and an opening for delivery of a gastric band at a distal end thereof. The introduction device also includes a transparent section formed at the distal end of the support tube.

It is also an object of the present invention to provide a gastric band introduction device wherein the distal end includes a transparent distal tube member.

It is another object of the present invention to provide a gastric band introduction device wherein the support tube is coextruded or molded.

It is a further object of the present invention to provide a gastric band introduction device wherein the transparent section extends only partially about a circumference of the support tube.

It is also another object of the present invention to provide a gastric band introduction device including an inner rod having a proximal end and a distal end, the inner rod is located within the support tube and adapted to slide therein.

It is still another object of the present invention to provide a gastric band introduction device wherein the inner rod includes a thumb ring at a proximal end and one or more upwardly protruding pins for engaging a gastric band at the distal end thereof.

It is yet a further object of the present invention to provide a gastric band introduction device wherein the inner rod also includes a shelf located at the distal end thereof for holding the gastric band.

It is also an object of the present invention to provide a gastric band introduction device wherein the inner rod also includes a longitudinal protrusion for effecting the sliding engagement between the inner rod and the support tube and also for stabilizing the inner rod when it is inserted into the support tube.

It is another object of the present invention to provide a gastric band introduction device including a tether secured to the gastric band for controlling deployment thereof.

It is a further object of the present invention to provide a method for the application of a gastric band through the utilization of a gastric band introduction device. The method is achieved by removing the gastric band introduction device from its packaging, initiating a check of the gastric band prior to introduction within the patient, the check including viewing of the gastric band through a transparent section formed in the introduction device, and introducing the gastric band.

It is also an object of the present invention to provide a method wherein the gastric band is a balloon type gastric band and initiation of a check includes performing a leak test of the balloon-type gastric band.

It is another object of the present invention to provide a method wherein the leak test includes applying air pressure to the balloon-type gastric band and observing through the transparent section of the support tube whether the balloon inflates in a desired manner.

It is still another object of the present invention to provide a method wherein the leak test includes evacuating the gastric band prior to loading into the introduction device.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of an unactuated gastric band introduction device in accordance with the present invention.

FIG. 2 is a perspective view of an actuated gastric band introduction device as shown in FIG. 1.

FIG. 3 is a partial side view of the distal end of the inner rod of the gastric band introduction device.

FIG. 4 is a perspective view of an actuated gastric band introduction device with the gastric band partially removed from the inner rod.

FIG. 5 is an enlarged partial view of the inner rod with the gastric band partially removed.

FIG. 6 is a perspective view of an alternate embodiment of a gastric band introduction device.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

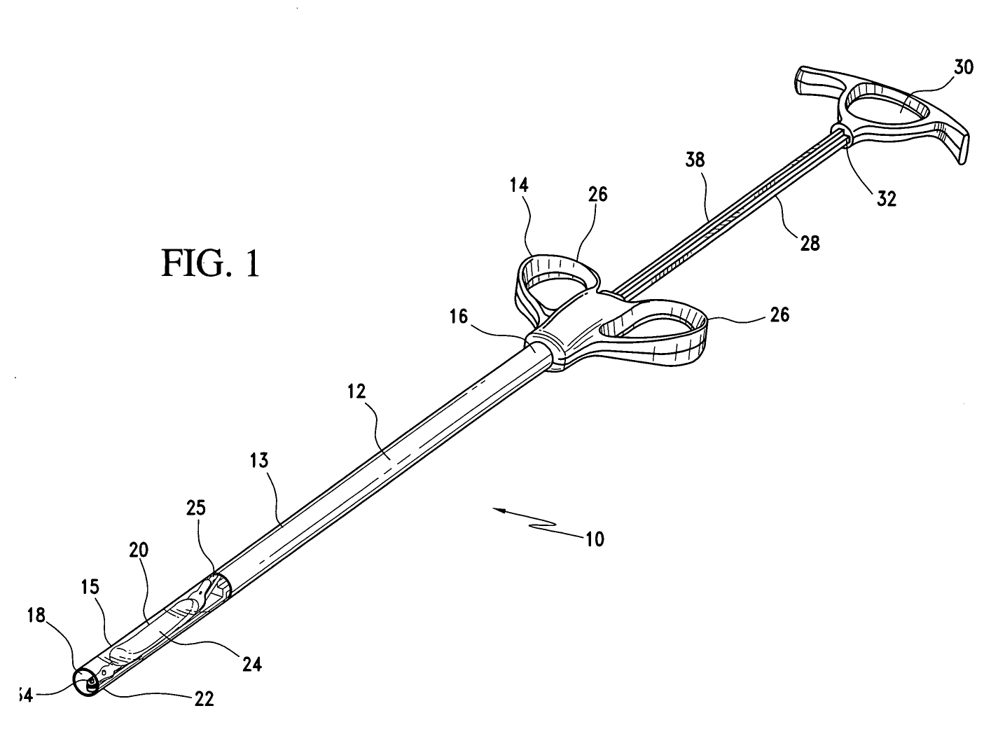

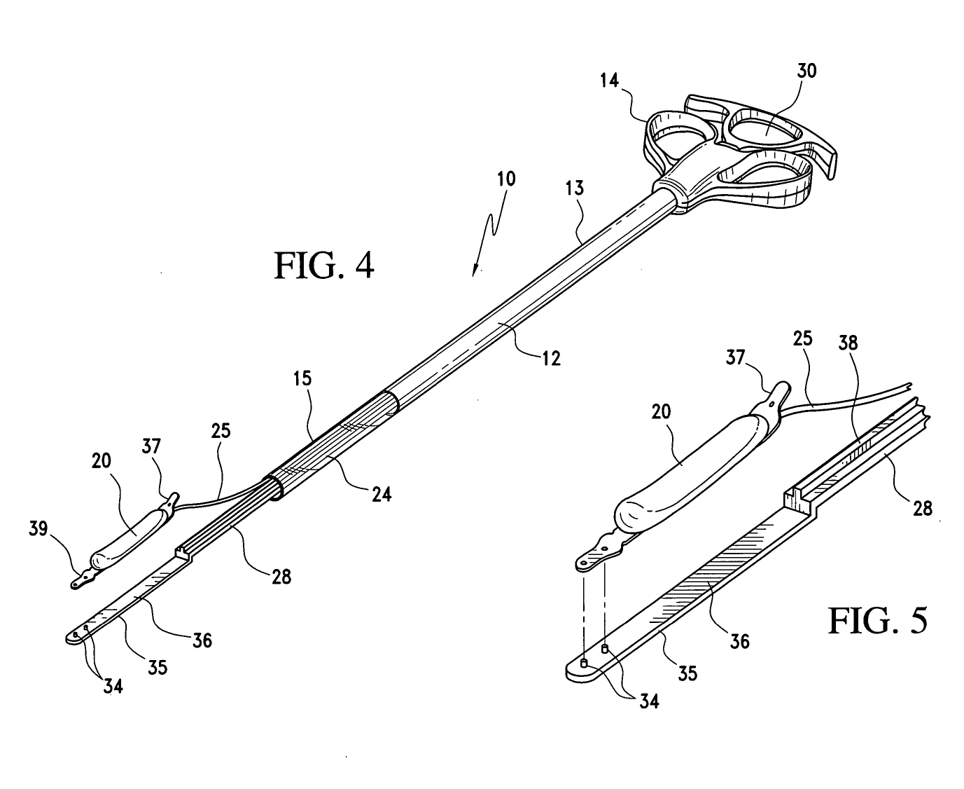

Referring to the FIGS. 1 to 5, a gastric band introduction device 10 is shown. The gastric band introduction device 10 includes an elongated support tube 12 having a handle 14 at a proximal end 16 thereof and an opening 18 for delivery of a gastric band 20 at a distal end 22 thereof. As will be discussed below in greater detail, the distal end 22 of the support tube 12 is provided with a transparent section 24 allowing viewing of the gastric band 20 stored therein. The gastric band 20 is held in position by a tether 25 extending up through the support tube 12 for control by the medical practitioner introducing the gastric band 20.

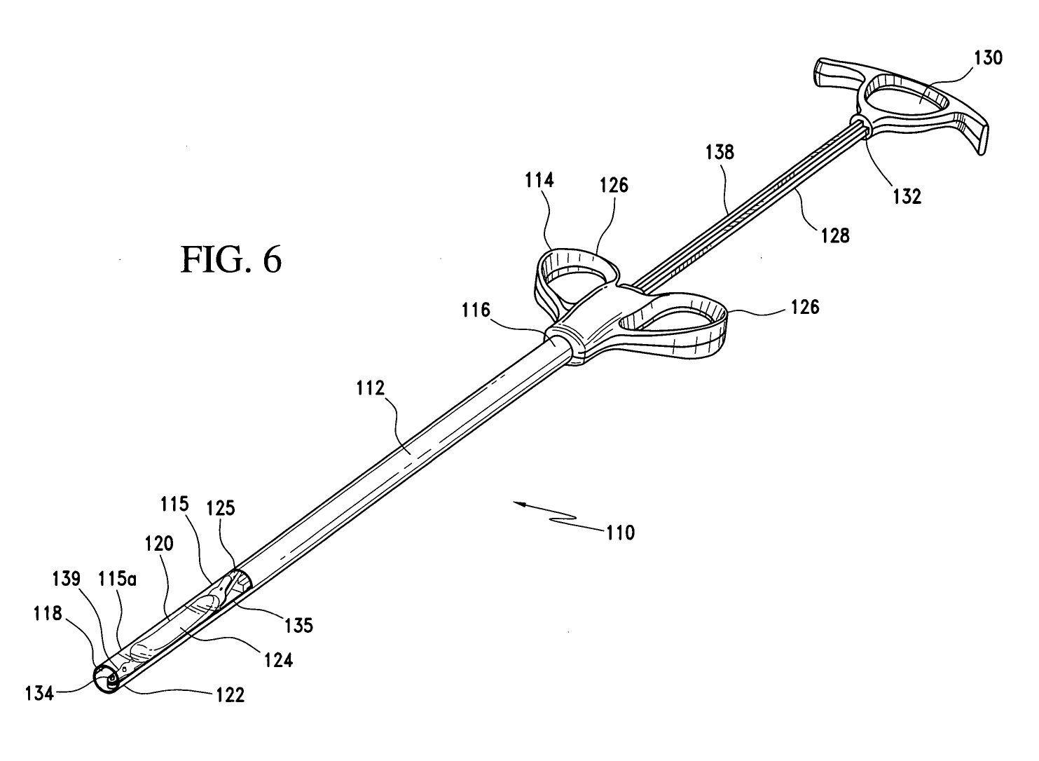

It is contemplated the transparent section may be formed in a variety of ways without departing from the spirit of the present invention. For example, and with reference to FIGS. 1 to 5, the support tube 12 may be formed from a proximal tube member 13 and a distal tube member 15 joined to define the complete support tube 12. The proximal tube member 13 may be formed from a variety of materials, while the distal tube member 15 is preferably a clear extruded tube. In accordance with this embodiment and as those skilled in the art will certainly appreciate, the joining of the proximal tube member 13 and the distal tube member 15 may be accomplished by a snap joint, glue joint or weld joint. Similarly the support tube could be coextruded or molded with the transparent portion of the tube positioned at the distal end. In addition, and reference to FIG. 6, the distal tube member 115 could be formed with only a portion 115a thereof being transparent (that is, half of the tube cross-section like a door). For example, the distal end 122 of the support tube 112 would be provided with an arcuate transparent section 115a that extends only partially about the circumference of the support tube 112.

Referring to FIG. 1, an isometric view of an unactuated gastric band introduction device 10 ready for introduction into a patient is presented. The gastric band introduction device 10 includes an elongated support tube 12 with a handle 14 at a proximal end 16. The gastric band introduction device 10 preferably includes a pair of opposed finger loops 26 extending outwardly from the handle 14. An inner rod 28 is located to slide within the support tube 12. The inner rod 28 includes a thumb ring 30 at a proximal end 32 thereof and one or more upwardly protruding pins 34 for engaging the gastric band 20 at a distal end 35 thereof. The inner rod 28 preferrably includes a shelf 36 located at the distal end 35 of the inner rod 28 for holding the gastric band 20. The inner rod 28 also includes a longitudinal protrusion 38 shaped and dimensioned for engaging a longitudinal groove (not shown) in the support tube 12 for effecting the sliding engagement between the inner rod 28 and support tube 12. The provision of the longitudinal protrusion and the longitudinal groove also function to stabilize the inner rod 28 when it is inserted into the support tube 12.

The support tube 12 is preferably comprised of a durable engineered plastic, although other materials may be used without departing from the spirit of the present invention. Preferably, the support tube 12 has a diameter of about 10 mm to about 20 mm and more preferably between about 12 mm and about 15 mm. Preferably, the support tube 12 is between about 30 cm and about 50 cm in length and more preferably about 43 cm. However, it should be appreciated that the support tube can be adapted in both length and diameter to accommodate any kind of gastric band or to fit within any sized trocar. The handle, the finger loops, the inner rod, and the thumb ring can be comprised of either stainless steel or any suitably durable engineering plastic. While pins are shown in the illustrative embodiment, any other mechanism, such as a clip or a strap, which will releasably secure the gastric band to the inner rod while also allowing the gastric band to be easily removed from the inner rod will suffice.

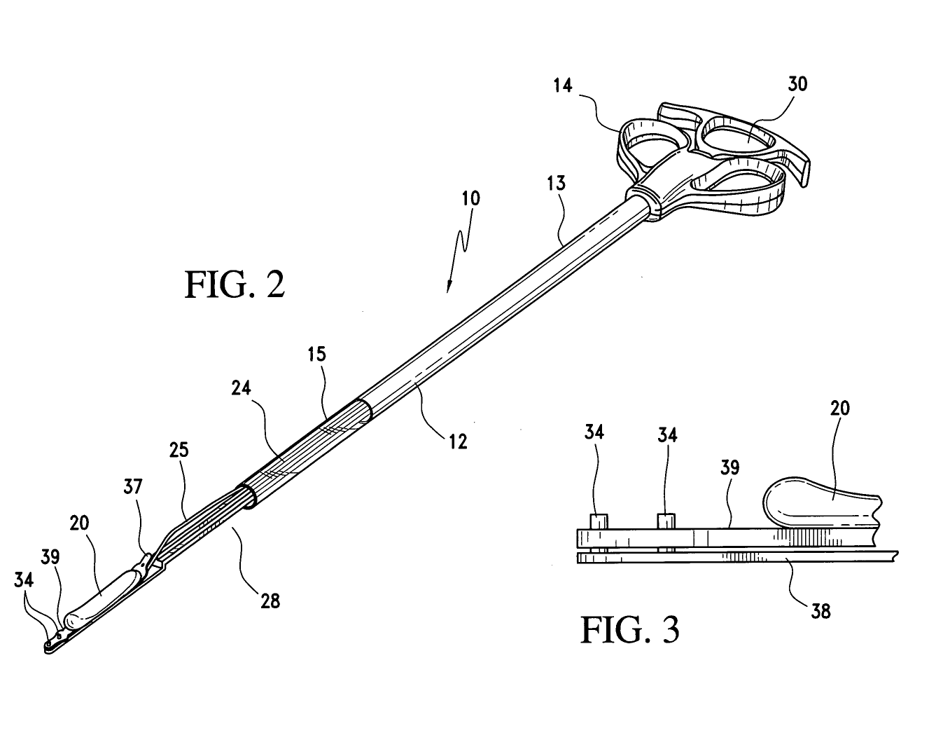

FIGS. 2 through 5 show the gastric band introduction device 10 after the device has been actuated to deploy a gastric band 20 from the distal end 22 of the support tube 12. In FIGS. 2 through 5, the inner rod 28 has been fully inserted into the support tube 12 and has pushed the gastric band 20 from the distal end 22 of the support tube 12. Once the gastric band 20 has been deployed, the gastric band 20 can be removed from the inner rod 28.

Generally, the gastric band 20 is delivered to a desired location via the utilization of the tether 25 to control the proximal end 37 of the gastric band 20 while the distal end 35 of the gastric band 20 is manipulated via a control arm releasably attached via clamp to the distal end 39 of the gastric band 20 for wrapping the gastric band about an individual's stomach in a well known manner.

In use, the medical practitioner removes the gastric band introduction device 10 from its packaging and initiates a check of the gastric band 20 prior to implantation within the patient. In particular, the medical practitioner will perform a leak test of the balloon-type gastric band 20. The leak testing procedure involves applying air pressure to the balloon-type gastric band 20 and observing whether the balloon inflates in a desired manner. Because of the transparent section 24 at the distal end 22 of the support tube 12, the medical practitioner need only look through the support tube 12 as the leak test is performed. It is contemplated that leak testing may be further enhanced by evacuating the gastric band 20 prior to loading into the support tube 12. As such, the medical practitioner will be instructed to look for partially inflation of the gastric band's bladder, which would be indicative of a leak allowing for the passage of air into the gastric band's bladder.

Once testing is completed, the gastric band introduction device 10, in an unactuated state, is inserted into a trocar placed in the patient's abdomen. The device 10 is then actuated by sliding and inserting the inner rod 28 fully into the support tube 12, thereby deploying the gastric band 20. The gastric band 20 is then removed from the inner rod 28 for placement around the stomach. Once the gastric band 20 has been removed, the inner rod 28 is pulled back out of the support tube 12, and the device is removed from the trocar.

In accordance with an alternate embodiment as shown in FIG. 6 and briefly discussed above, an alternate introduction device 110 may be utilized in delivering the gastric band 120 to a desired location. More particularly, the introduction device 110 includes an elongated support tube 112. As with the prior embodiment, the elongated support tube 112 includes a handle 114 at a proximal end 116 thereof and an opening 118 for delivery of a gastric band 120 at a distal end 122 thereof. The gastric band introduction device 110 also includes a pair of opposed finger loops 126 extending outwardly from the handle 114. An inner rod 128 is located to slide within the support tube 112. The inner rod 128 includes a thumb ring 130 at a proximal end 132 thereof and one or more upwardly protruding pins 134 for engaging the distal end 139 of the gastric band 120. The inner rod 128 preferrably includes a shelf located at the distal end 135 of the inner rod 128 for holding the gastric band 120. The inner rod 128 also includes a longitudinal protrusion 138 shaped and dimensioned for engaging a longitudinal groove (not shown) in the support tube 112 for effecting the sliding engagement between the inner rod 128 and support tube 112. The provision of the longitudinal protrusion and the longitudinal groove also function to stabilize the inner rod 128 when it is inserted into the support tube 112.

As with the prior embodiment, the distal end 122 of the support tube 112 is provided with a transparent section 124 allowing viewing of the gastric band 120 stored therein. The gastric band 120 is positioned at the distal end 122 of the support tube 112 and is presented for viewing based upon the transparent section 124 at the distal end 122 thereof. However, and in accordance with the embodiment, the transparent section 124 only constitutes a portion of the arc defined by the support tube 112 at the distal end 124 thereof. More particularly, the distal end 122 of the support tube 112 is provided with an arcuate transparent section 115a that extends only partially about the circumference of the support tube 112. The gastric band 120 is held in position by a tether 125 extending up through the support tube 112 for control by the medical practitioner introducing the gastric band 120.

As with the prior embodiment, the support tube 112 is preferably comprised of a durable engineered plastic, although other materials may be used without departing from the spirit of the present invention. The dimensions of the support tube 112 are similar to those discussed above and may be varied to accommodate any kind of gastric band or to fit within any size trocar.

In use, this embodiment is employed in a manner substantially similar to the embodiment disclosed with reference to FIGS. 1 to 5 and described above in detail.

It will become readily apparent to those skilled in the art that the above invention has equally applicability to other types of implantable bands. For example, bands are used for the treatment of fecal incontinence. One such band is described in U.S. Pat. No. 6,461,292 which is hereby incorporated herein by reference. Bands can also be used to treat urinary incontinence. One such band is described in U.S. Patent Application 2003/0105385 which is hereby incorporated herein by reference. Bands can also be used to treat heartburn and/or acid reflux. One such band is described in U.S. Pat. No. 6,470,892 which is hereby incorporated herein by reference. Bands can also be used to treat impotence. One such band is described in U.S. Patent Application 2003/0114729 which is hereby incorporated herein by reference.

While the present invention has been illustrated by the description of several embodiments and while the illustrative embodiments have been described in considerable detail, it is not the intention of the applicant to restrict or in any way limit the scope of the appended claims to such detail. Additional advantages and modifications may readily appear to those skilled in the art.

While the preferred embodiments have been shown and described, it will be understood that there is no intent to limit the invention by such disclosure, but rather, is intended to cover all modifications and alternate constructions falling within the spirit and scope of the invention.

1. A gastric band introduction device, comprising:
an elongated support tube having a handle at a proximal end thereof and an opening for delivery of a gastric band at a distal end thereof;
a transparent section formed at the distal end of the support tube.

2. The gastric band introduction device according to claim 1, wherein the distal end includes a transparent distal tube member.

3. The gastric band introduction device according to claim 1, wherein the support tube is coextruded or molded.

4. The gastric band introduction device according to claim 1, wherein the transparent section extends only partially about a circumference of the support tube.

5. The gastric band introduction device according to claim 1, further including an inner rod having a proximal end and a distal end, the inner rod is located within the support tube and adapted to slide therein.

6. The gastric band introduction device according to claim 5, wherein the inner rod includes a thumb ring at a proximal end and one or more upwardly protruding pins for engaging a gastric band at the distal end thereof.

7. The gastric band introduction device according to claim 6, wherein the inner rod further includes a shelf located at the distal end thereof for holding the gastric band.

8. The gastric band introduction device according to claim 7, wherein the inner rod further includes a longitudinal protrusion for effecting the sliding engagement between the inner rod and the support tube and also for stabilizing the inner rod when it is inserted into the support tube.

9. The gastric band introduction device according to claim 1, further including a tether secured to the gastric band for controlling deployment thereof.

10. A method for the application of a gastric band through the utilization of a gastric band introduction device, comprising:
removing the gastric band introduction device from its packaging;
initiating a check of the gastric band prior to introduction within the patient, the check including viewing of the gastric band through a transparent section formed in the introduction device; and
introducing the gastric band.

11. The method according to claim 10, wherein the gastric band is a balloon type gastric band and initiation of a check includes performing a leak test of the balloon-type gastric band.

12. The method according to claim 11, wherein the leak test includes applying air pressure to the balloon-type gastric band and observing through the transparent section of the support tube whether the balloon inflates in a desired manner.

13. The method according to claim 11, wherein the leak test includes evacuating the gastric band prior to loading into the introduction device.
